(19) United States
(12) Patent Application Publication
(10) Pub. No.: US 2018/0259603 A1
(43) Pub. Date: Sep. 13, 2018

(54) RADIO FREQUENCY ANTENNA ASSEMBLY FOR MAGNETIC RESONANCE IMAGE GUIDED THERAPY

(71) Applicant: KONINKLIJKE PHILIPS N.V., EINDHOVEN (NL)

(72) Inventors: CHRISTOPH LEUSSLER, EINDHOVEN (NL); CHRISTIAN FINDEKLEE, EINDHOVEN (NL)

(21) Appl. No.: 15/762,217

(22) PCT Filed: Sep. 13, 2016
(86) PCT No.: PCT/EP2016/071477

§ 371 Date: Mar. 22, 2018

(30) Foreign Application Priority Data
Sep. 25, 2015 (EP) 15186963.3

(51) Int. Cl. G01R 33/3415; G01R 33/34; G01R 33/345; G01R 33/48; G01R 33/565; A61N 5/10

(52) U.S. Cl. CPC G01R 33/3415; G01R 33/34007; G01R 33/3456; A61N 2005/1087; G01R 33/5659; A61N 5/1049; A61N 2005/1055; G01R 33/481

(57) ABSTRACT

The radio frequency (RF) antenna assembly has sets of antenna conductors that leave an opening between the sets. A radiotherapy beam path may pass through the opening so that the antenna conductors are at most minimally exposed to the radiation. Each set of antenna conductors has a surface conductor loop and a transverse conductor loop. The surface conductor loop is arranged on cylindrical surface and generates an RF field mostly in its axial range. The transverse conductor loop extends radially and generates an RF field in the axial range of the opening. In this way a homogeneous RF field within the RF antenna assembly.

### FIELD OF THE INVENTION

The invention pertains to a radio frequency antenna assembly for a magnetic resonance examination system, notably for a magnetic resonance image guided therapy system.

Magnetic resonance imaging (MRI) methods utilize the interaction between magnetic fields and nuclear spins in order to form two-dimensional or three-dimensional images are widely used nowadays, notably in the field of medical diagnostics, because for the imaging of soft tissue they are superior to other imaging methods in many respects, do not require ionizing radiation and are usually not invasive.

According to the MRI method in general, the body of the patient to be examined is arranged in a strong, uniform magnetic field B₀ whose direction at the same time defines an axis (normally the z-axis) of the co-ordinate system to which the measurement is related. The magnetic field B₀ causes different energy levels for the individual nuclear spins in dependence on the magnetic field strength which can be excited (spin resonance) by application of an electromagnetic alternating field (RF field) of defined frequency (so-called Larmor frequency, or MR frequency). From a macroscopic point of view the distribution of the individual nuclear spins produces an overall magnetization which can be deflected out of the state of equilibrium by application of an electromagnetic pulse of appropriate frequency (RF pulse) while the corresponding magnetic field B₁ of this RF pulse extends perpendicular to the z-axis, so that the magnetization performs a precession motion about the z-axis. The precession motion describes a surface of a cone whose angle of aperture is referred to as flip angle. The magnitude of the flip angle is dependent on the strength and the duration of the applied electromagnetic pulse. In the example of a so-called 90° pulse, the magnetization is deflected from the z axis to the transverse plane (flip angle 90°).

After termination of the RF pulse, the magnetization relaxes back to the original state of equilibrium, in which the magnetization in the z direction is built up again with a first time constant T1 (spin lattice or longitudinal relaxation time), and the magnetization in the direction perpendicular to the z-direction relaxes with a second and shorter time constant T2 (spin-spin or transverse relaxation time). The transverse magnetization and its variation can be detected by means of receiving RF antennae (coil arrays) which are arranged and orientated within an examination volume of the magnetic resonance examination system in such a manner that the variation of the magnetization is measured in the direction perpendicular to the z-axis. The decay of the transverse magnetization is accompanied by dephasing taking place after RF excitation caused by local magnetic field inhomogeneities facilitating a transition from an ordered state with the same signal phase to a state in which all phase angles are uniformly distributed. The dephasing can be compensated by means of a refocusing RF pulse (for example a 180° pulse). This produces an echo signal (spin echo) in the receiving coils.

In order to realize spatial resolution in the subject being imaged, such as a patient to be examined, constant magnetic field gradients extending along the three main axes are superposed on the uniform magnetic field B0, leading to a linear spatial dependency of the spin resonance frequency. The signal picked up in the receiving antennae (coil arrays) then contains components of different frequencies which can be associated with different locations in the body. The signal data obtained via the receiving coils correspond to the spatial frequency domain of the wave-vectors of the magnetic resonance signal and are called k-space data. The k-space data usually include multiple lines acquired of different phase encoding. Each line is digitized by collecting a number of samples. A set of k-space data is converted to an MR image by means of Fourier transformation.

The transverse magnetization dephases also in presence of constant magnetic field gradients. This process can be reversed, similar to the formation of RF induced (spin) echoes, by appropriate gradient reversal forming a so-called gradient echo. However, in case of a gradient echo, effects of main field inhomogeneities, chemical shift and other off-resonances effects are not refocused, in contrast to the RF refocused (spin) echo.

### BACKGROUND OF THE INVENTION

A radio frequency coil for a magnetic resonance examination system is known from the U.S. Pat. No. 4,680,548.

The known radio frequency coil is a high-pass version birdcage coil made up of two conductive loop elements electrically interconnected by axially conductive segments. The loop elements include serially connected capacitors and the loop elements have inherent inductances. The known birdcage coil is operated in a quadrature excitation mode in which the birdcage coil transmits a circularly polarised radio frequency magnetic field which interacts with magnetic spins in the subject to be examined, notably a patient to be examined.

### SUMMARY OF THE INVENTION

An object of the invention is to provide a radio frequency antenna assembly for a magnetic resonance image guided therapy system.

This object is achieved by the radio frequency antenna assembly comprising

a plurality of sets of antenna conductors arranged on a cylindrical surface and said sets being arranged in groups that are mutually axially offset leaving an axially and angularly extending opening between the groups of sets of antenna conductors

each of the sets of antenna conductors including a surface conductor loop having its area in the cylindrical angular and axial directions and

at least one transverse conductor loop having its area extending radially with respect to the cylindrical surface.

The therapy system generally is configured for irradiating a target zone in the body of the patient to be treated. Such irradiation generally involves a high-energy radiation beam, such as an x-ray beam, γ-beam, proton beam or a high-intensity ultrasound beam to deposit energy into the target zone. The therapy system may be combined with a magnetic resonance examination system into an MR image guided therapy system. The MR image guided therapy system functions to provide image guidance to the orientation of the therapeutic radiation beam onto a target zone to be treated. The magnetic resonance examination system is provided with a radio frequency antenna assembly to generate the dynamic electromagnetic fields in an examination zone. The circularly polarised magnetic field component is employed to manipulate the spins, for example to excite spins transversely to the main magnetic field directions and for refocusing or inverting spins. The RF antenna assembly can be operated in a transmit mode to apply the dynamic electromagnetic fields and in a receive mode to receive magnetic resonance signals. Electronic and structural components of radio frequency (RF) antennae, such as the known birdcage coil often are susceptible to radiation damage caused by the radiation when that it passed through the structure of the RF antenna. The electronics and structural components of RF antennae generally have a low resistivity against the radiation beam. Notably, electronics components are quite vulnerable to radiation damage. The radio frequency (RF) antenna assembly of the present invention provides for an opening between the groups of sets of antenna conductors through with the radiation beam can pass and no structural or electronics components of the RF antenna are located in the radiation beam's path. The opening is formed by said groups of sets of antenna conductors being mutually axially offset. In this way an axial opening between the groups is left through which the therapeutic radiation beam may pass. Hence, radiation damage to electronics and structural components of the RF antenna assembly is avoided. Moreover, there is no need to shield or protect the structural and electronics components from the radiation beam. Further, the radiation beam is hardly or not at all perturbed by the structural and electronics components of the RF antenna assembly. This adds to accurately directing the radiation beam onto the target zone. The opening between the groups of sets of antenna conductors of the RF antenna assembly may extend axially as well as angularly. The axial width of the opening allows room for the beam path of the radiation beam of the therapy system. In another example, a PET detector may be accommodated in the opening. In that example, the axial and angular ranges of the opening are selected to provide sufficient space in the opening for the PET-detectors. This example of the RF antenna assembly is suitable for a combined PET-MRI system.

In the RF antenna assembly of the present invention the configuration of antenna elements in sets with the surface conductor in the cylindrical surface and the transverse conductor loop that extends radially achieves a good homogeneity of the radio frequency (RF) field within the RF antenna assembly, notably in the axial range and angular range of the opening. This is notably achieved by the transverse conductor loops in each of the sets of antenna conductors of which the emitted field has a relative large axial range. The surface conductors contribute to the field within the RF antenna assembly surrounding the axial range of the opening. The surface conductor on the cylindrical surface generates an RF field component predominantly in the axial range of the surface conductor and this RF field component decreases axially in the opening. On the other hand, the transverse conductor loop generates an RF field component that extends substantially axially into the opening. In practice the transverse conductor loop is configured such that its RF field component does not decrease more than by a factor of two from the axial edge of the opening to the axial centre of the opening. Thus, the RF antenna assembly of the invention has a very good spatial homogeneity of the dynamic radio frequency magnetic (B₁) field within the RF antenna assembly, notably over the axial range of the opening which adds to improve the image quality of the magnetic resonance images on the basis of which the radiation beam is controlled to be directed precisely at the target zone.

The angular range of the opening determines the orientation range of the direction of the radiation beam. An angular range of 2π allows the target zone to be irradiated from all angular directions. The opening may have a more limited angular range. When the opening extends only over a limited angular range, then outside of that angular range, the surface conductors may cover the axial dimension and better field homogeneity within the RF antenna assembly is achieved. For example, an angular range of 2π is required for MR Linac, as the radiation passes the body and at the opposite end, the radiation is absorbed by a radiation load. For MR HIFU the opening's angular range can be it so an opening is only required for posterior coil. This may also apply for various interventional applications, where the interventionalist requires an opening on the top side to access the patient's body. Further an option is to have several openings say four openings with 45° angular range that could be applied for magnetic resonance and positron emission tromography (MR PET) to accommodate PET-detectors in the openings, in this implementation between some of the coils of the RF antenna arrangement may not be separated by the opening and the field homogeneity in the opening would be improved.

In summary, the radiofrequency (RF) antenna assembly of the invention has sets of antenna conductors that leave an opening between the sets. A radiotherapy beam path may pass through the opening so that the antenna conductors are at most minimally exposed to the radiation. Each set of antenna conductors has a surface conductor loop and a transverse conductor loop. The surface conductor loop is arranged on cylindrical surface and generates an RF field mostly in its axial range. The transverse conductor loop extends radially and generates an RF field in the axial range of the opening. In this way a homogeneous RF field is generated within the RF antenna assembly.

These and other aspects of the invention will be further elaborated with reference to the embodiments defined in the dependent Claims.

In an example of the RF antenna assembly of the invention, the surface conductors may be surface coils or surface strips arranged in the cylindrical surface. The transverse conductor loops may be transverse coil loops. In another example of the RF antenna assembly of the invention the surface conductor is a cylindrical area section and the transverse conductor loop is a transverse protrusion section. The transverse protrusion section is connected to the cylindrical area section. The cylindrical area section is arranged axially on the cylindrical surface and the transverse protrusion section extends at an angle, preferably radially, from the cylindrical surface. The closer the transverse protrusion section is orientated radially, the stronger the transverse protrusion section extends into the opening. The cylindrical area and the transverse protrusion section may together form a single electrically conducting loop.

In a further embodiment of the invention individual sets of antenna conductors include several transverse coil loops associated with each surface conductors. For example an individual set may include one, two, three or more transverse coil loops and a single surface conductor. The more transverse loops per single surface conductor in an individual set of antenna conductors are employed, the better the spatial field homogeneity. Very good results are achieved when two or three transverse coil loops are employed. Adding more transverse loops in a set of antenna conductors increases the complexity of the configuration but does not improve the field homogeneity much more. Additional field contribution and better signal sensitivity and field homogeneity are achieved using several transverse coil loops. These transverse coil loops can be run in parallel as individually decoupled transmit/receive coils. For example, each individual cylindrical area coil may be associated with at least one transverse coil. These coils can be decoupled by usual decoupling techniques. Optionally also be a mix of cylindrical area section without transverse protrusion and cylindrical area section with a transverse protrusion may be employed.

Preferably, the transverse coil loop extends axially up to the edge of the opening. The field of the transverse coil loop extends well into the axial direction into the opening and contributes to the field homogeneity in the axial and angular ranges of the opening at a high power efficiency.

Preferably the surface conductors extend axially beyond the transverse coil loops at the axial ends. In other words, the transverse coil loop's axial extension does not reach the axial ends of the RF antenna assembly. In this configuration strong stray fields are avoided axially outside the RF antenna assembly. This configuration also improves the power efficiency, because no RF field is generated where that is not needed. In fact, in this configuration the RF field decreases over a short axial range outside of the RF antenna assembly. For example the centre B1 field strength drops to 50% in the transverse plane at the end of coil conductor.

In a further embodiment the transverse coil loops of the sets of antenna conductors of the RF assembly are circuited to operate as a TEM resonator. This is advantageous notably at radiofrequencies exceeding 200 MHz, where TEM coils produce a more uniform RF field distribution. In this embodiment the RF coil assembly is provided with a radio frequency (RF) screen. The RF screen is placed radially outward from the cylindrical surface in which the sets of antenna conductors are located. The transverse coil loops are electrically connected to the RF screen, so that the RF screen provides a return path for the electrical currents introduced into the transverse coil loops. The return conductor, which has a larger distance from the patient can be a wider strip, i.e. the return conductor strip's width is larger than the strip that functions as the antenna element transmitting the RF field. This provides a better RF shielding. The electrically conducting strips are easily and inexpensively manufactured using printed-circuit board technology and design. The return conductor, which has a larger distance from the examination zone in which the patient to be examined is positioned can be a wider strip, thus providing a better RF shielding. When the antenna is used as a transmit/receive antenna, then a larger RF shield is considered to prevent radiation and coupling to the arms of the patient. Low inductivity means that propagation effects on the conductor are reduced, thus lower electrical fields and lower losses and higher SNR. Notably, strips of 0.5-1.0 cm width are employed as the transmitting antenna elements, while wider strips of 5-10 cm width are employed as return conductors. When the antenna is used as a transmit/receive antenna, then a larger RF shield is considered to prevent radiation and coupling to the arms of the patient. Low inductivity means that propagation effects on the conductor are reduced, thus lower electrical fields and lower losses. This is due to a uniform phase distribution over the conductors.

In yet a further embodiment, the surface conductors are formed as electrically conducting strips. These electrically conducting strips are connected to the RF screen, so as to provide a current return path for the electrical currents that are passed through the electrically conducting strips. This embodiment of the RF antenna assembly has a low inductivity.

In another embodiment an RF antenna arrangement is provided that has an anterior and a posterior RF antenna assembly. Each of the anterior and posterior RF antenna assembly have their antenna conductors arranged on respective cylindrical surfaces. The radius of curvature of these respective cylindrical surfaces may be different. This geometry adds to make efficient use of bore space and take better account of the cross sectional shape of the patient to be treated. Preferably, the anterior RF antenna assembly has an opening between its sets of antenna conductors as specified in Claim 1. Optionally, the posterior RF antenna may have an opening between its groups of sets of antenna conductors as specified in Claim 1. These opening(s) allow the radiation beam to pass to the target zone, without having to pass the antenna conductors are electronic components of the RF antenna assembly(ies). In another embodiment, the antenna conductors are arranged on a flexible carrier that forms the cylindrical surface that is deformable or flexible to fit the patient to be treated. Preferably, the antenna conductors themselves are rigid, and mounted on a flexible substrate, such as a carrier or a former. the RF antenna arrangement can be shaped by flexing the carrier with the antenna conductors on it. As the antenna conductors themselves are not deformed, there is no need for (extensive) re-tuning of the RF antenna arrangement upon deformation.

These and other aspects of the invention will be elucidated with reference to the embodiments described hereinafter and with reference to the accompanying drawing wherein

### BRIEF DESCRIPTION OF THE DRAWINGS

FIG. 1 shows a three-dimensional schematic view of an embodiment of the RF antenna assembly of the invention,

FIGS. 2 and 3 show a schematic views of examples of a set of antenna conductors for the RF antenna assembly of the invention FIGS. 4 and 5 show a schematic views of an another example of several sets of antenna conductors for the RF antenna assembly of the invention,

FIGS. 6 and 7 show diagrammatic representation if the RF field distribution of examples of sets of antenna elements of the RF antenna assemblies of the invention and

FIG. 8 shows a schematic front view of a magnetic resonance examination system in which the RF antenna assembly of the invention is incorporated

FIG. 9 shows a diagrammatic representation of an magnetic resonance examination system in which the invention is incorporated.

### DETAILED DESCRIPTION OF THE EMBODIMENTS

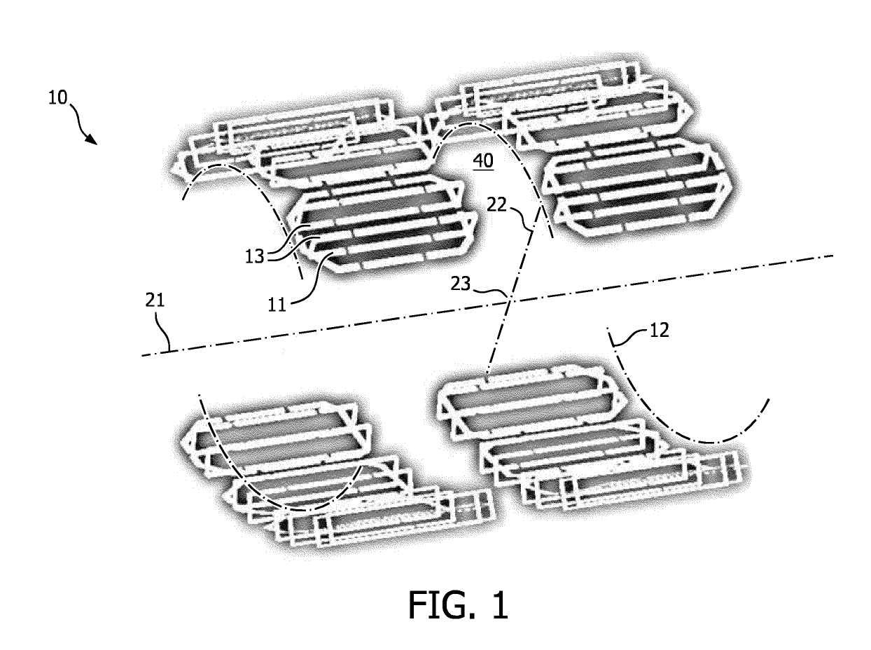

FIG. 1 shows a three-dimensional schematic view of an embodiment of the RF antenna assembly of the invention. The RF antenna assembly of the invention has a number of sets 10 of antenna conductors. The antenna conductors form the antenna elements that constitute the resonant structure. The sets of antenna conductors 10 leave the angularly and axially extending opening 40. The opening is left between groups of the sets of antenna elements. In the example shown there are twelve of these sets of antenna conductors 11,13; six sets are arranged at either sides of the opening 40. Each of these six sets forms a group of sets of antenna conductors. The sets are axially displaced so as to leave the opening between them. These sets of antenna elements are arranged on a cylindrical surface 12 that is schematically partially indicated in FIG. 1. The cylindrical surface may be a cylindrical coil former that carries the antenna conductors. The cylinder axis 21 is orientated axially. When in use in an magnetic resonance examination system the cylinder axis is orientated along the direction of the magnetic resonance examination system's main magnetic field. the sets of antenna conductors are radially 22 apart so as to leave an examination zone 23 within the cylindrical surface. When in use, the RF antenna assembly can generate the dynamic RF (B₁) field in the examination zone. When in use in an MR image guide therapy system, a therapeutic radiation beam, such as a beam of γ-rays, high-energy x-rays, protons or high-intensity focused ultrasound passes through the opening 40 towards a target region of a patient to be treated in the examination zone 23. To that end the RF antenna assembly of the invention is mounted in the MR image guided system such that the beam path of the therapeutic radiation beam passes through the opening.

The antenna conductors of the RF antenna assembly of the invention are configured to generate a spatially homogeneous B₁-field distribution in the examination zone, while there are no antenna conductors in the axially and angularly extending opening. Each of the sets of antenna conductors includes a surface conductor loop 11 with one or more transverse conductor loops 13. In the example shown in FIG. 1, two transverse conductor loops are associated with each single surface conductor. The surface conductor loop is a flat conductor loop that is arranged generally in the cylindrical surface. The surface conductor loop generates the B₁-field in the examination zone in the axial and angular range over which the surface conductor loop extends and along a radial range towards the cylinder axis. The transverse conductor loop extends radially and generates a B₁-field component that extends axially from the transverse coil conductor. The B₁-field contribution from the surface conductor loop and the transverse coil conductor(s) of the sets of antenna conductors combine to a spatially uniform B₁-field distribution in the examination zone. The B₁-field in the examination zone is controlled by controlling the (phase and amplitude, frequency, time, waveform) of AC electrical currents applied to the surface conductor loops and the transverse conductor loops. Most detailed control is achieved by independent control of electrical currents to each individual surface and each transverse conductor loop. Quite detailed control is achieved when electrical currents to each of the sets of antenna conductors is independently controlled.

As shown in FIG. 1, the surface conductor loop is formed as axially elongate conductor loop having its loop area in the cylinder surface. The transverse conductor loops are each transverse conductor loops that extend radially and axially. The transverse conductor loop have their loop areas extending radially and extend axially elongate.

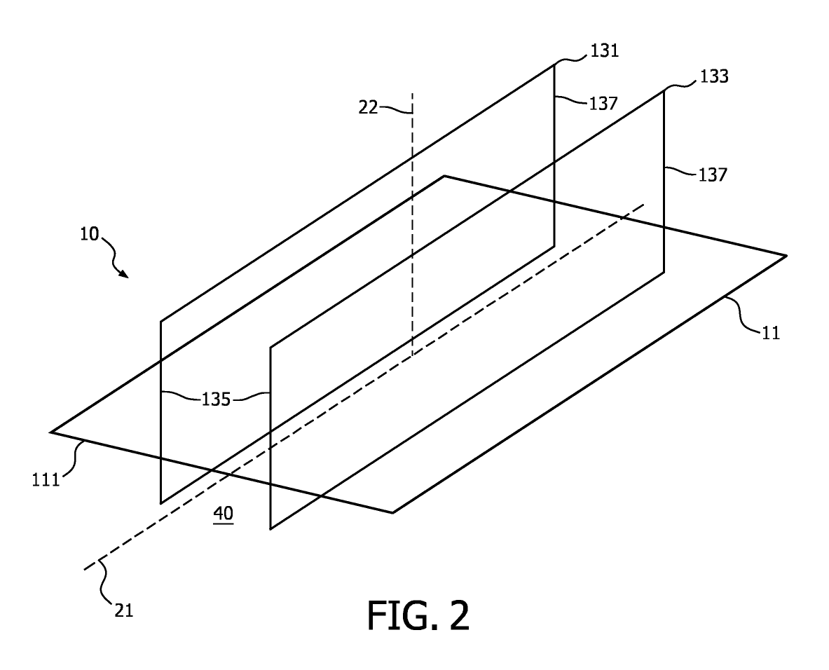

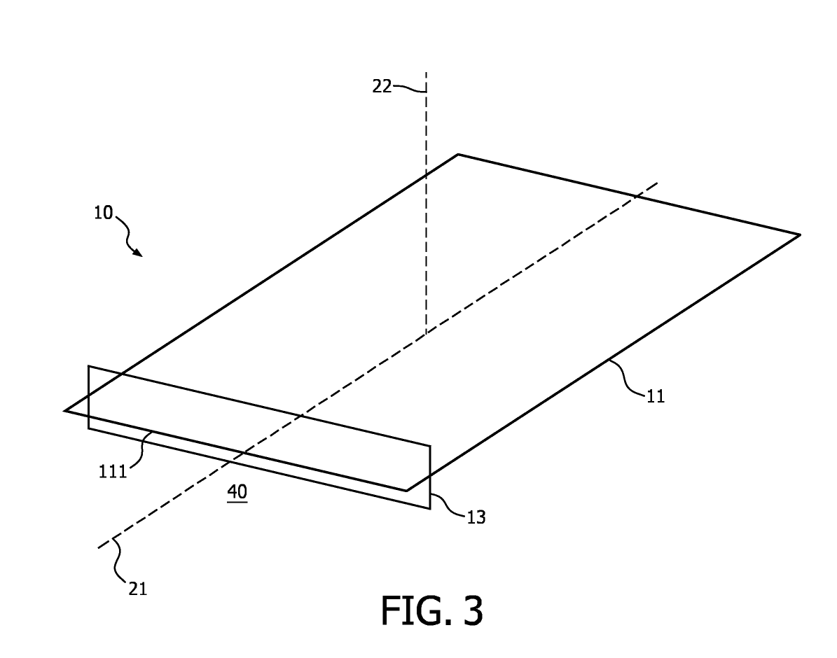

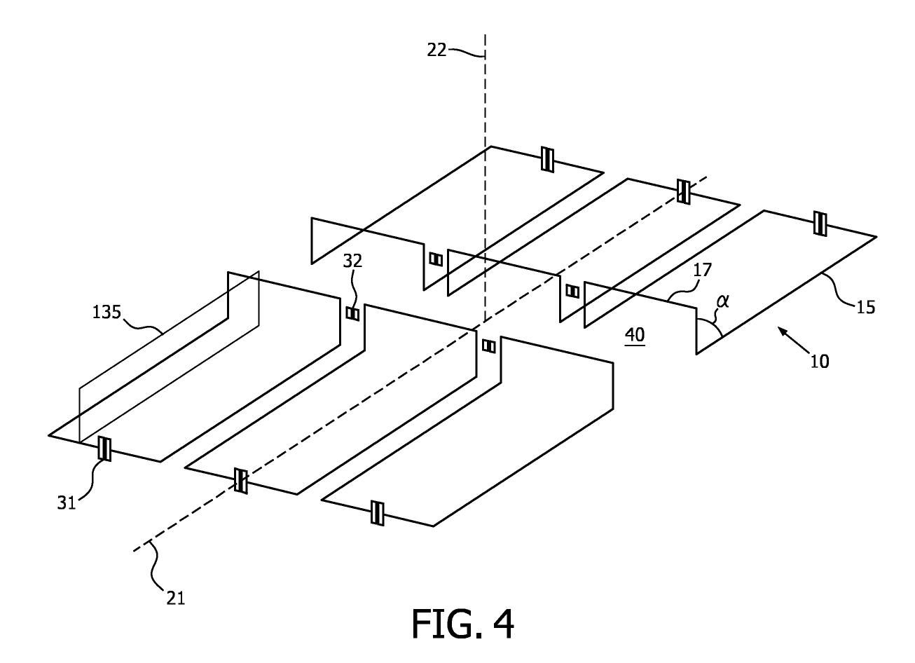

FIGS. 2 and 3 show a schematic views of examples of a set of antenna conductors for the RF antenna assembly of the invention. FIG. 2 shows an example of a set 10 of antenna conductors with one surface conductor loop 11 and two transverse coil conductors 131, 133. The transverse conductor loop 131,133 extends axially all the way up to the axial boundary of the opening 40. That is, the boundary of the opening is formed by the angular conductor 111 and the radial conductors 135 at the side of the opening 40. At the axial end of the transverse coil conductor facing away from the opening 40, the surface conductor loop 11 extends axially beyond the axial extension of the transverse coil conductor 131 133. That is, the transverse coil conductors do not extend all the way to the axial end of the RF antenna assembly. Thus, the B₁-field component of the transverse coil conductor extends well into the opening to contribute the B1-field in axial range of the opening 40, while the transverse coil conductor does not generate an appreciable B₁-field component extending axially outside of the RF antenna assembly. Thus, no undesired stray B₁-field is produced and the power efficiency of the RF antenna assembly is improved.

In FIG. 3, an example of a set of antenna conductors for the RF antenna assembly of the invention is shown. Here the transverse conductor loop 13 is a transverse conductor loop that is elongate along the angular direction of the cylinder surface of the RF antenna assembly and extends radially. The transverse conductor loop is located at the edge of the opening 40, i.e. near the angular conductor 111 of the surface coil loop 11. This transverse conductor loop predominantly generates its B₁-field into the axial direction into the opening 40. Decoupling of the transverse conductor loops is performed by capacitive and/or inductive decoupling. Capacitive decoupling is realized by connecting the protrusion sectors in parallel and using a common capacitor. Inductive decoupling. can be realized in a similar approach.

FIG. 4 shows a schematic view of an another example of several sets of antenna conductors for the RF antenna assembly of the invention. In this version each set is formed by a single conductor loop having a cylindrical area section 15 that is arranged in the cylinder surface and a transverse protrusion section 17 that extends at an angle to the area of the cylindrical area section 15. This single conductor loop may be combined with a transverse conductor loop 135 as shown with only one of the single conductor loops in FIG. 4. Preferably the transverse protrusion is along the radial direction, i.e. perpendicular to the area of the cylindrical area section. The transverse protrusion may be transverse to the cylindrical area section at an angle α in the range of 60°-120°, while very good results are achieved when α=90°. This set of antenna conductors is formed from a single conductor loop and is easy to manufacture. Because only the electrical current to each set of conductors, viz. the single conductor loop is applied, the electrical currents to each of the sets of antenna elements is simple to control. The transverse protrusion section generates its B₁-field component that extents predominantly into the axial range of the opening 40. The cylindrical area section generates its B₁-field component predominantly along the axial range of the elongate cylindrical area section. In order to achieve proper resonant properties, tuning capacitors 31 are provided in the cylindrical area sections 15. Decoupling capacitors 32 link adjacent protrusion sections for capacitive decoupling of the protrusion sections 17.

In the embodiment of FIG. 5, the decoupling inductances 42 inductively decouple the protrusions 17. Decoupling inductances 32 link adjacent protrusions for inductively decoupling the protrusions.

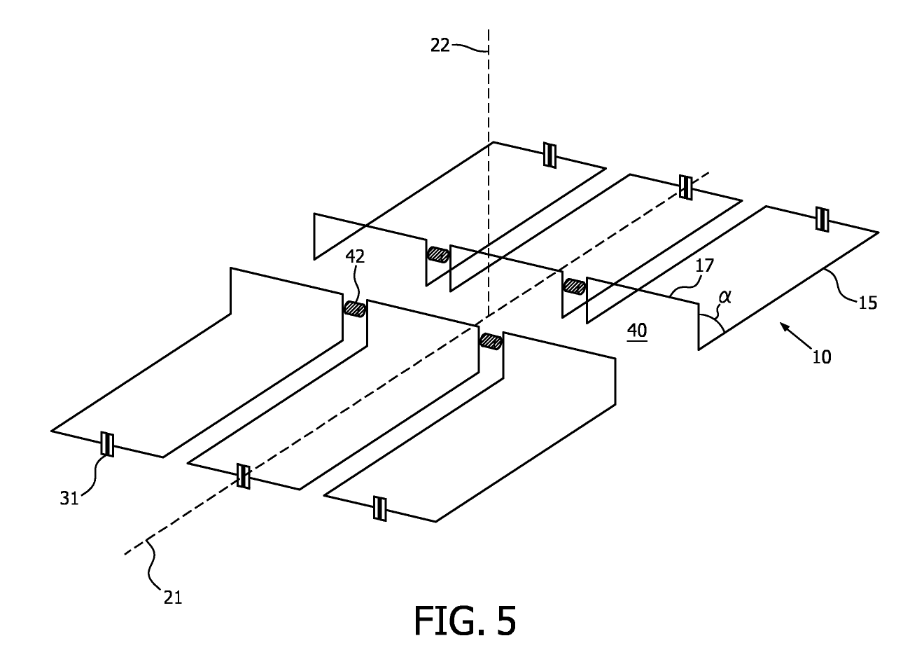

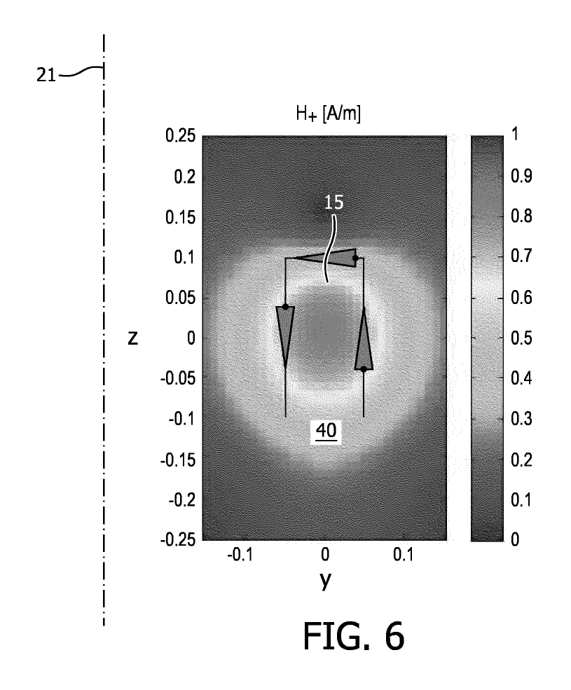

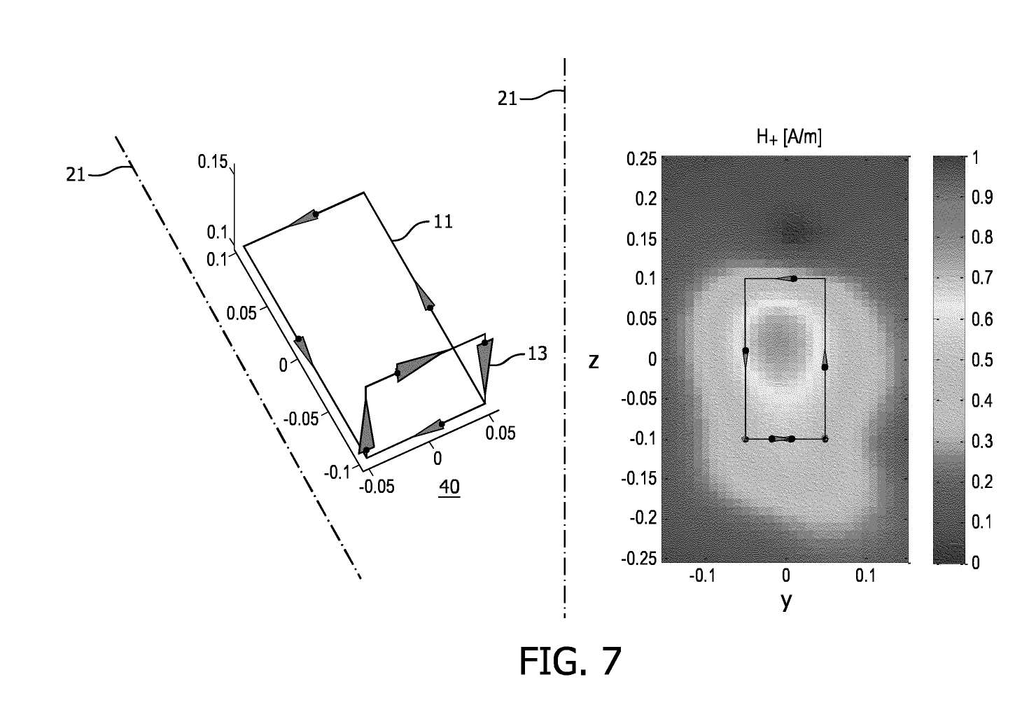

FIGS. 6 and 7 show diagrammatic representations of the RF field distribution of examples of sets of antenna elements of the RF antenna assemblies of the invention. FIG. 6 shows diagrammatically the B₁-field distribution in the angular and radially directions of one of the sets of antenna conductors 15,17 of FIG. 4. FIG. 6 shows a diagrammatic representation of the B1-field distribution in a lateral plane through the cylinder axis 21 in a plane 10 cm below the coil loop. FIG. 6 shows that the B₁-field extends axially into the opening 40 at negative z-values). At the opposite axial end of the cylindrical area section 15 (positive z-values) the B₁-field extends hardly or not at all beyond the axial extension of the RF antenna assembly. FIG. 7 shows diagrammatically the B₁-field distribution in the angular and radially directions of one of the sets of antenna conductors 15,17 of FIG. 3. The phase of the electrical current applied to the surface conductor loop 11 and the transverse conductor loop 13 can be adjusted. In this particular simulation the electrical current in the vertical, smaller transverse loop 13 was 3.75 times that of the larger surface coil loop 11. The lateral deviation of the field generation inside the gap appears to be controlled by the current ratio and the phase of the current intended. Allowing one more degree of freedom by changing also the phase the current ratio (more difficult to realize), this can be improved as shown in FIG. 7. This field plot was generated by choosing amplitude ratio of 3.75, together with the phase ratio of 45°.

The B₁-field is shown to extend into the opening 40. The angular distribution of the B₁-field, notably, the predominant axis 120 along which the B₁-field extends from the set of conductors 10 is orientated with respect to the axial direction is determined by the phase difference between the electrical current applied to the surface conductor loop and the transverse conductor loop.

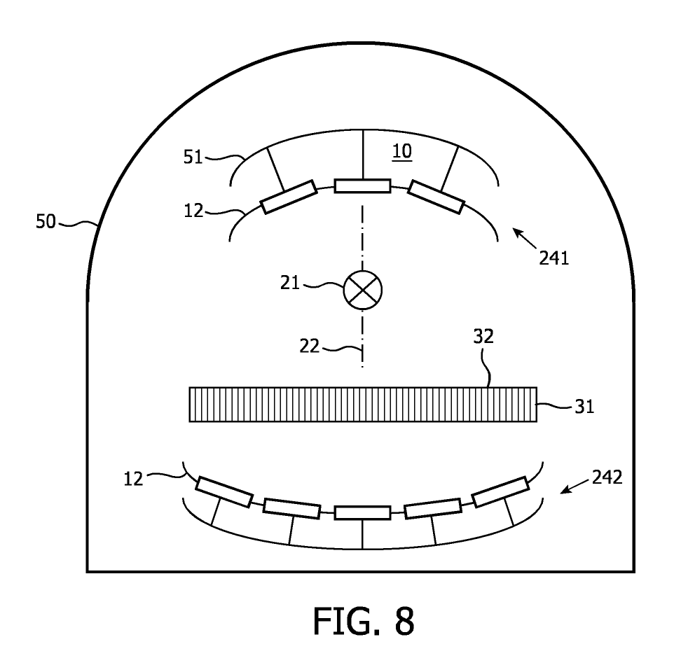

FIG. 8 shows a schematic front view of a magnetic resonance examination system in which the RF antenna assembly of the invention is incorporated. The magnetic resonance examination system includes a gantry 50 in which the magnet system with a main magnet and gradient system are mounted. In the gantry 50 the patient carrier 31, such as a patient table is mounted. The patient carrier has a support surface 32 on which a patient to be examined (and/or treated) is positioned. The patient carrier is axially moveable along the axis 21. The axis 21 is along the direction of the main magnetic field. The RF antenna element has an anterior 241 and posterior 242 antenna assembly located at opposite sides of the patient carrier. The anterior RF antenna assembly is mounted at the side of the support surface 32 onto which the patient to be examined is placed. The posterior RF antenna assembly is located at the opposite side of the patient carrier, generally that is underneath the patient carrier 31. The cylinder surface 12 of the anterior RF antenna assembly and of the posterior RF antenna assembly have different radius of curvature. This allows to make efficient use of the available bore space in the main magnet. Further, the RF assembly is configured in this way to correspond to the cross-sectional shape of the patient. This correspondence can be further improved by disposing the sets of antenna elements on a flexible carrier.

In the example of FIG. 8, the surface conductors 11 of the sets 10 of antenna conductors are formed as electrically conducting strips that are axially orientated. The RF antenna assembly further comprises a radio frequency (RF) screen located radially from the sets of antenna elements. The electrically conducting strips are coupled to the RF screen which provides for a return current path. This coupling may be galvanic, inductive or capacitive. The RF antenna elements with eh RF screen are electrically circuited as a TEM resonator.

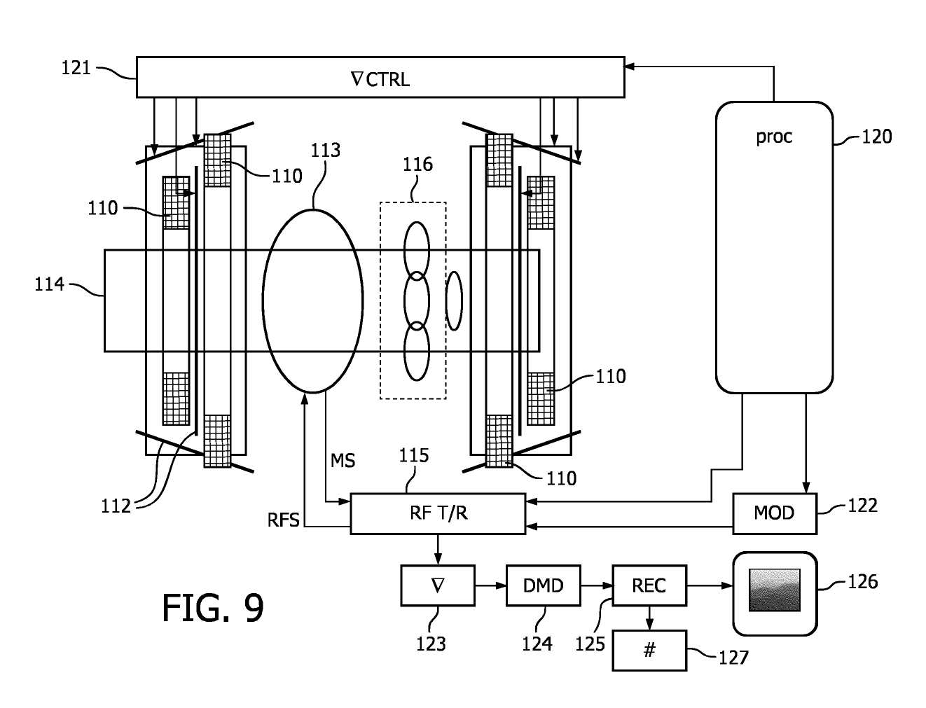

FIG. 9 shows diagrammatically more details of a magnetic resonance imaging system in which the invention is used. The magnetic resonance imaging system includes a main magnet with a set of main coils 10 whereby the steady, uniform magnetic field is generated. The main coils are constructed, for example in such a manner that they from a bore to enclose a tunnel-shaped examination space. The patient to be examined is placed on a patient carrier which is slid into this tunnel-shaped examination space. The magnetic resonance imaging system also includes a number of gradient coils 111, 112 whereby magnetic fields exhibiting spatial variations, notably in the form of temporary gradients in individual directions, are generated so as to be superposed on the uniform magnetic field. The gradient coils 111, 112 are connected to a gradient control 121 which includes one or more gradient amplifier and a controllable power supply unit. The gradient coils 111, 112 are energised by application of an electric current by means of the power supply unit 121; to this end the power supply unit is fitted with electronic gradient amplification circuit that applies the electric current to the gradient coils so as to generate gradient pulses (also termed ‘gradient waveforms’) of appropriate temporal shape. The strength, direction and duration of the gradients are controlled by control of the power supply unit. The magnetic resonance imaging system also includes transmission and receiving antennae (coils or coil arrays) 113, 116 for generating the RF excitation pulses and for picking up the magnetic resonance signals, respectively. The transmission coil 113 is preferably constructed as a body coil 13 whereby (a part of) the object to be examined can be enclosed. The body coil is usually arranged in the magnetic resonance imaging system in such a manner that the patient 30 to be examined is enclosed by the body coil 113 when he or she is arranged in the magnetic resonance imaging system. The body coil 13 acts as a transmission antenna for the transmission of the RF excitation pulses and RF refocusing pulses. Preferably, the body coil 113 involves a spatially uniform intensity distribution of the transmitted RF pulses (RFS). The same coil or antenna is generally used alternately as the transmission coil and the receiving coil. Typically, a receiving coil includes a multiplicity of elements, each typically forming a single loop. Various geometries of the shape of the loop and the arrangement of various elements are possible The transmission and receiving coil 113 is connected to an electronic transmission and receiving circuit 115.

It is to be noted that is that there is one (or a few) RF antenna elements that can act as transmit and receive; additionally, typically, the user may choose to employ an application-specific receive antenna that typically is formed as an array of receive-elements. For example, surface coil arrays 116 can be used as receiving and/or transmission coils. Such surface coil arrays have a high sensitivity in a comparatively small volume. The receiving coil is connected to a preamplifier 123. The preamplifier 123 amplifies the RF resonance signal (MS) received by the receiving coil 116 and the amplified RF resonance signal is applied to a demodulator 124. The receiving antennae, such as the surface coil arrays, are connected to a demodulator 124 and the received pre-amplified magnetic resonance signals (MS) are demodulated by means of the demodulator 124. The pre-amplifier 123 and demodulator 124 may be digitally implemented and integrated in the surface coil array The demodulated magnetic resonance signals (DMS) are applied to a reconstruction unit. The demodulator 124 demodulates the amplified RF resonance signal. The demodulated resonance signal contains the actual information concerning the local spin densities in the part of the object to be imaged. Furthermore, the transmission and receiving circuit 115 is connected to a modulator 122. The modulator 122 and the transmission and receiving circuit 115 activate the transmission coil 113 so as to transmit the RF excitation and refocusing pulses. In particular the surface receive coil arrays 116 are coupled to the transmission and receive circuit by way of a wireless link. Magnetic resonance signal data received by the surface coil arrays 116 are transmitted to the transmission and receiving circuit 115 and control signals (e.g. to tune and detune the surface coils) are sent to the surface coils over the wireless link.

The reconstruction unit derives one or more image signals from the demodulated magnetic resonance signals (DMS), which image signals represent the image information of the imaged part of the object to be examined. The reconstruction unit 125 in practice is constructed preferably as a digital image processing unit 125 which is programmed so as to derive from the demodulated magnetic resonance signals the image signals which represent the image information of the part of the object to be imaged. The signal on the output of the reconstruction is applied to a monitor 126, so that the reconstructed magnetic resonance image can be displayed on the monitor. It is alternatively possible to store the signal from the reconstruction unit 125 in a buffer unit 127 while awaiting further processing or display.

The magnetic resonance imaging system according to the invention is also provided with a control unit 120, for example in the form of a computer which includes a (micro)processor. The control unit 120 controls the execution of the RF excitations and the application of the temporary gradient fields. To this end, the computer program according to the invention is loaded, for example, into the control unit 120 and the reconstruction unit 125.

1. A radio frequency antenna assembly comprising
a plurality of sets of antenna conductors arranged in groups on a cylindrical surface and said groups of sets being mutually axially offset leaving an axially and angularly extending opening between the sets of antenna conductors
each of the sets of antenna conductors including a surface conductor loop having its area in the cylindrical angular and axial directions and
at least one transverse conductor loop having its area extending radially with respect to the cylindrical surface.

2. A radio frequency antenna assembly as claimed in claim 1, wherein in individual sets of antenna conductors a cylindrical area section forming the surface conductor loop and connected to a transverse protrusion section forming the transverse conductor loop are provided, where the cylindrical area section is arranged on the cylinder surface and the transverse protrusion section extends radially.

3. A radio frequency antenna assembly as claimed in claim 2, where in the cylindrical area section and the transverse protrusion section form a single electrically conducting loop.

4. A radio frequency antenna assembly as claimed in claim 1, wherein in individual sets of antenna conductors two transverse coil loops are associated with the surface conductor loop.

5. A radio frequency antenna assembly as claimed in claim 1, wherein the transverse coil loop extends axially up to the opening.

6. A radio frequency antenna assembly as claimed in claim 1, wherein the surface conductor extends axially beyond the transverse coil loop towards the RF antenna assembly's axial ends.

7. A radio frequency antenna assembly as claimed in claim 1, wherein the transverse coil loops from a TEM resonator.

8. A radio frequency antenna assembly as claimed in claim 1, wherein the surface conductor loop is a surface coil loop.

9. A radio frequency antenna assembly as claimed in claim 1, further comprising a RF screen and wherein the transverse coil loop is an electrically conducting strip that is electrically connected to the RF screen.

10. A radio frequency antenna arrangement comprising an anterior radio frequency antenna assembly as claimed in claim 1, and a posterior radio frequency antenna, wherein the radius of curvature of the cylinder surface of the posterior radio frequency assembly is different form the radius of curvature of the cylinder surface of the anterior radio frequency assembly.

11. A magnetic resonance examination system comprising a patient carrier with a support face, wherein a radio frequency antenna assembly of claim 1 is mounted at the patient carrier's side opposite its support face, or is integrated in the patient carrier.

12. A magnetic resonance examination system as claimed in claim 11, in which the radio frequency assembly is mounted to or integrated moveably with respect to the patient carrier.
